(19) United States
(12) Patent Application Publication
(10) Pub. No.: US 2022/0254028 A1
(43) Pub. Date: Aug. 11, 2022

(54) METHOD AND APPARATUS FOR ADJUSTING BLOOD FLOW VELOCITY IN MAXIMUM HYPEREMIA STATE BASED ON INDEX FOR MICROCIRCULATORY RESISTANCE

(71) Applicant: SUZHOU RAINMED MEDICAL TECHNOLOGY CO., LTD., Suzhou (CN)

(72) Inventors: Guangzhi LIU, Suzhou (CN); Yanjun GONG, Suzhou (CN); Jianping LI, Suzhou (CN); Tieci YI, Suzhou (CN); Bo ZHENG, Suzhou (CN)

(73) Assignee: SUZHOU RAINMED MEDICAL TECHNOLOGY CO., LTD., Suzhou (CN)

(21) Appl. No.: 17/731,607

(22) Filed: Apr. 28, 2022

Related U.S. Application Data
(63) Continuation of application No. PCT/CN2019/116674, filed on Nov. 8, 2019.

(30) Foreign Application Priority Data
Nov. 4, 2019 (CN) 201911065694.6

(51) Int. Cl. G06T 7/00; A61B 5/026; A61B 34/10; G06T 7/11; G06T 7/13; G06T 17/00

(52) U.S. Cl. CPC G06T 7/0016; A61B 5/026; A61B 34/10; G06T 7/11; G06T 2210/41; G06T 17/00; A61B 2034/105; G06T 2207/30104; G06T 2207/30172; G06T 7/13

(57) ABSTRACT

Provided are a method and apparatus for adjusting blood flow velocity in maximum hyperemia state based on index for microcirculatory resistance. The method comprises: acquiring an index for microcirculatory resistance iFMR during a diastolic phase according to a blood flow velocity v, an aortic pressure waveform, and an physiological parameter (S100); making an adjustment parameter r equal to 1 if the index for microcirculatory resistance iFMR during the diastolic phase is less than K; making the adjustment parameter r satisfy a formula r=1−(iFMR−K)/100 if the index for microcirculatory resistance iFMR during the diastolic phase is greater than or equal to K, wherein K is a positive number less than 100 (S200); acquiring a corrected blood flow velocity in a maximum hyperemia state according to a product of the adjustment parameter and a blood flow velocity in the maximum hyperemia state (S300).

### CROSS REFERENCE TO RELATED APPLICATIONS

This application is a continuation of International Patent Application No. PCT/CN2019/116674 filed on Nov. 8, 2019 which claims the benefit of priority from the Chinese Patent Application No. The disclosure claims priority to Chinese Patent Application No. 201911065694.6 filed before Chinese National Intellectual Property Administration on Nov. 4, 2019, entitled “METHOD AND APPARATUS FOR ADJUSTING BLOOD FLOW VELOCITY IN MAXIMUM HYPEREMIA STATE BASED ON INDEX FOR MICROCIRCULATORY RESISTANCE”, the entire contents of which are hereby incorporated by reference.

### TECHNICAL FIELD

The present disclosure relates to the field of coronary artery technology, and in particular, to a method and an apparatus for adjusting blood flow velocity in maximum hyperemia state based on index for microcirculatory resistance, a coronary artery analysis system and a computer storage medium.

### BACKGROUND

According to the statistics of the World Health Organization, cardiovascular diseases have become a “leading killer” of human health. In recent years, the analysis of the physiological and pathological behaviors of cardiovascular diseases using hemodynamics has also become a very important means of diagnosis of the cardiovascular diseases.

Blood flow quantity and flow velocity are very important parameters of hemodynamics. How to measure the blood flow quantity and flow velocity accurately and conveniently has become the focus of many researchers.

Due to different vital signs of different populations, evaluation standards for normal values are slightly different. For example, the myocardial microcirculation function of the elderly is relatively poor, and the blood flow velocity is generally lower than that of the young. If the industry general evaluation standard is used, the blood flow velocity used will be higher than the actual value. The higher blood flow velocity will further affect the coronary artery evaluation parameters, such as: fractional flow reserve FFR, fractional flow reserve during a diastolic phase iFR, and index for microcirculatory resistance iFMR during the diastolic phase.

Therefore, at this stage, how to obtain the index for microcirculatory resistance iFMR according to individual differences, and then adjust the blood flow velocity in the maximum hyperemia state according to the index for microcirculatory resistance iFMR, to obtain a more targeted blood flow velocity with individualized differences and improve the accuracy of the blood flow velocity, has become an urgent problem in the field of coronary artery technology.

### SUMMARY

The present disclosure provides a method and an apparatus for adjusting blood flow velocity in maximum hyperemia state based on index for microcirculatory resistance, a coronary artery analysis system and a computer storage medium, so as to solve the problem of how to obtain a more targeted, individualized blood flow velocity in the maximum hyperemia state according to individualized differences.

In order to achieve the above object, in a first aspect, the present disclosure provides a method for adjusting blood flow velocity in maximum hyperemia state based on index for microcirculatory resistance, comprising:

acquiring an index for microcirculatory resistance iFMR during a diastolic phase according to a blood flow velocity v, an aortic pressure waveform, and an physiological parameter;

making an adjustment parameter r equal to 1 if the index for microcirculatory resistance iFMR during the diastolic phase is less than K;

making the adjustment parameter r satisfy a formula

$$r = 1 - \frac{iFMR - K}{100}$$

if the index for microcirculatory resistance iFMR during the diastolic phase is greater than or equal to K, wherein K is a positive number less than 100;

acquiring a corrected blood flow velocity in a maximum hyperemia state according to a formula v′=rvₕ;

wherein v′ represents the corrected blood flow velocity in the maximum hyperemia state, and vₕ represents a blood flow velocity in the maximum hyperemia state.

Optionally, in the above method for adjusting blood flow velocity in maximum hyperemia state based on index for microcirculatory resistance,

vₕ=zv̅+x

wherein vₕ represents the blood flow velocity in the maximum hyperemia state, v̅ represents an average blood flow velocity in a heartbeat cycle area, z is a constant in the range of 1 to 3, and x is a constant in the range of 50 to 300; K=50.

Optionally, in the above method for adjusting blood flow velocity in maximum hyperemia state based on index of microcirculatory resistance, a manner for acquiring an index for microcirculatory resistance iFMR during a diastolic phase according to a blood flow velocity v, an aortic pressure waveform, and an physiological parameter comprises:

selecting a maximum value of the blood flow velocity v, i.e., a maximum blood flow velocity vₘₐₓ during the diastolic phase;

a time period corresponding to the vₘₐₓ being the diastolic phase, acquiring an average aortic pressure during the diastolic phase according to the aortic pressure waveform;

$$iFMR = \overline{{P}_{a}} / {v}_{max} \times k + c ;$$

$$\overline{{P}_{a}} = \frac{{\sum }_{1}^{j} ( {P}_{a 1} + {P}_{a 2} … {P}_{aj} )}{j} ;$$

wherein, P̅ₐ̅ represents the average aortic pressure during the diastolic phase; Pₐ₁, Pₐ₂, and Pₐⱼ represent aortic pressures corresponding to a first point, a second point, and a j-th point within the diastolic phase on the aortic pressure waveform, respectively, and j represents the number of pressure points contained in the aortic pressure waveform during the diastolic phase, vₕ represents the blood flow velocity in the maximum hyperemia state obtained by selecting a maximum value from all blood flow velocities v; k and c represent the influence parameters k=1˜3, c=0˜10.

Optionally, in the above method for adjusting blood flow velocity in maximum hyperemia state based on index for microcirculatory resistance, the influence parameter k=a×b, wherein a represents a characteristic value of diabetes, b represents a characteristic value of hypertension, and c represents gender.

Optionally, in the above method for adjusting blood flow velocity in maximum hyperemia state based on index for microcirculatory resistance, if a patient does not suffer from diabetes, then 0.5≤a≤1; if the patient suffers from diabetes, then 1<a≤2;

if the patient's blood pressure is greater than or equal to 90 mmHg, then 1<b≤1.5; if the patient's blood pressure is less than 90 mmHg, then 0.5≤b≤1;

if the patient is male, then c=0; if the patient is female, then c=3˜10.

Optionally, in the above method for adjusting blood flow velocity in maximum hyperemia state based on index for microcirculatory resistance, if the patient does not suffer from diabetes, then a=1; If the patient suffers from diabetes, then a=2;

if the patient's blood pressure is greater than or equal to 90 mmHg, then b=1.5; if the patient's blood pressure is less than 90 mmHg, then b=1;

if the patient is male, c=0; if the patient is female, c=5.

Optionally, in the above method for adjusting blood flow velocity in maximum hyperemia state based on index for microcirculatory resistance. a manner for acquiring a blood flow velocity comprises:

reading a group of two-dimensional coronary artery angiogram images of at least one body position;

extracting a blood vessel segment of interest from the group of two-dimensional coronary artery angiogram images;

extracting a centerline of the blood vessel segment;

determining a difference in time taken for a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL;

solving the blood flow velocity according to a ratio of ΔL to Δt.

Optionally, in the above method for adjusting blood flow velocity in maximum hyperemia state based on index for microcirculatory resistance, a manner for extracting a blood vessel segment of interest from the group of two-dimensional coronary artery angiogram images comprises:

selecting N frames of the two-dimensional coronary artery angiogram images from the group of two-dimensional coronary artery angiogram images;

acquiring the blood vessel segment of interest by picking a beginning point and an ending point of the blood vessel of interest on the two-dimensional coronary artery angiogram images.

Optionally, in the above method for adjusting blood flow velocity in maximum hyperemia state based on index for microcirculatory resistance, a manner for extracting the centerline of the blood vessel segment comprises:

extracting a blood vessel skeleton from the two-dimensional coronary artery angiogram images;

according to the extension direction of the blood vessel segment and the principle of obtaining the shortest path between two points;

extracting the centerline of the blood vessel segment along the blood vessel skeleton.

Optionally, in the above method for adjusting blood flow velocity in maximum hyperemia state based on index for microcirculatory resistance. a manner for determining a difference in time taken for a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL, and solving the blood flow velocity according to the ratio of ΔL to Δt comprises:

taking the coronary angiogram image when the contrast agent flows to the inlet of the coronary artery, that is, the beginning point of the blood vessel segment as a first frame of image, and taking the coronary angiogram image when the contrast agent flows to the ending point of the blood vessel segment as a N-th frame of image:

solving the time difference and centerline length difference of the N-th frame of image and a (N−1)th frame, . . . , a (N−b)th frame, . . . , a (N−a)th frame, . . . , the first frame of image, successively, with the time differences being Δt₁, . . . , Δr_{b}, . . . , Δtₐ, . . . , Δt_{N-1}, respectively; the centerline length differences being ΔL₁, . . . , ΔL_{b}, . . . , ΔLₐ, . . . , ΔL_{N-1}, respectively;

according to v=ΔL/Δt, obtaining the blood flow velocity from the N-th frame of image to the (N−1)th frame, . . . , the (N−b)th frame, . . . , the (N−a)th frame, . . . , the first frame of image, respectively, wherein v represents the blood flow velocity, with the blood flow velocity being v₁, . . . , v_{b}, . . . , V_{N-}, respectively.

Optionally, in the above method for adjusting blood flow velocity in maximum hyperemia state based on index for microcirculatory resistance, a manner for determining a difference in time taken a contrast agent flowing through the blood vessel segment in any two frames of the two-dimensional coronary artery angiogram images with the difference being Δt and determining a difference in centerline length of a sub-segment of the blood vessel segment through which the contrast agent flows in the two frames of two-dimensional coronary artery angiogram image with the difference being ΔL, and solving the blood flow velocity according to the ratio of ΔL to Δt comprises:

solving the time difference and centerline length difference of the N-th frame and b-th frame, of the (N−1)th frame and (b−1)th frame, . . . , of the (N−b−a)th frame and (N−a)th frame, . . . , of the (N−b+1)th frame and first frame of image, successively;

according to v=ΔL/Δt, obtaining the blood flow velocity from the N-th frame to the b-th frame, from the (N−1)th frame to the (b−1)th frame, . . . , from the (N−b−a)th frame to the (N−a)th frame, from the (N−b+1)th frame to the first frame of image, respectively, wherein v represents the blood flow velocity.

Optionally, in the above method for adjusting blood flow velocity in maximum hyperemia state based on index for microcirculatory resistance, after the manner for extracting a centerline of the blood vessel segment, and before the manner for determining a difference in time taken for a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL, the method further comprises:

reading a group of two-dimensional coronary artery angiogram images of at least two body positions;

acquiring geometric structure information of the blood vessel segment;

performing graphics processing on the blood vessel segment of interest:

extracting a blood vessel contour line of the blood vessel segment;

according to the geometric structure information of the blood vessel segment, synthesizing a three-dimensional blood vessel model by projecting the at least two body positions' two-dimensional coronary angiogram images which have been extracted centerline and contour line of the blood vessel onto a three-dimensional plane.

Optionally, in the above method for adjusting blood flow velocity in maximum hyperemia state based on index for microcirculatory resistance, a manner for solving the blood flow velocity according to the ratio of ΔL to Δt comprises:

according to the three-dimensional blood vessel model, acquiring a centerline of the three-dimensional blood vessel model, correcting the centerline extracted from the two-dimensional coronary angiogram images, and correcting the centerline difference ΔL to obtain ΔL′;

solving the blood flow velocity v according to the ratio of the ΔL′ to the Δt.

In a third aspect. the present disclosure provides an apparatus for adjusting blood flow velocity in maximum hyperemia state based on index for microcirculatory resistance, used for the above method for adjusting blood flow velocity in maximum hyperemia state based on index for microcirculatory resistance, comprising: a blood flow velocity acquisition unit, an aortic pressure waveform acquisition unit , a physiological parameter acquisition unit , an unit of index for microcirculatory resistance during diastolic phase and an adjustment parameter unit; the unit of index for microcirculatory resistance during diastolic phase is connected with the blood flow velocity acquisition unit, the aortic pressure waveform acquisition unit and the physiological parameter acquisition unit;

the blood flow velocity acquisition unit is configured to acquire a blood flow velocity v;

the aortic pressure waveform acquisition unit is configured to acquire, in real time, an aortic pressure waveform changing over time;

the physiological parameter acquisition unit is configured to acquire physiological parameters of a patient, comprising gender and disease history;

the unit of index for microcirculatory resistance during diastolic phase is configured to receive the blood flow velocity v, the aortic pressure waveform, and the physiological parameters sent by the blood flow velocity acquisition unit, the aortic pressure waveform acquisition unit, and the physiological parameter acquisition unit, and then to obtain an index for microcirculatory resistance iFMR during a diastolic phase according to the blood flow velocity v, the aortic pressure waveform, and the physiological parameters;

the adjustment parameter unit is configured to receive iFMR value of the unit of index for microcirculatory resistance during diastolic phase; make an adjustment parameter r equal to 1 if the index for microcirculatory resistance during the diastolic phase iFMR<K; make the adjustment parameter r to satisfy

a formula

$$r = 1 - \frac{iFMR - K}{100}$$

if the index for microcirculatory resistance during the diastolic phase iFMR≥K; where K is a positive number less than 100.

Optionally, the above apparatus for adjusting blood flow velocity in maximum hyperemia state based on index for microcirculatory resistance further comprises: an image reading unit, a blood vessel segment extraction unit, and a centerline extraction unit connected in sequence, a time difference unit and the physiological parameter acquisition unit both connected to the image reading unit, and the blood flow velocity acquisition unit that respectively connected with the time difference unit and a centerline difference unit, respectively; the centerline difference unit is connected with the centerline extraction unit;

the image reading unit is configured to read a group of two-dimensional coronary artery angiogram image of at least one body position;

the blood vessel segment extraction unit is configured to receive two-dimensional coronary artery angiogram images sent by the image reading unit, and to extract a blood vessel segment of interest in the images;

the centerline extraction unit is configured to receive the blood vessel segment sent by the blood vessel segment extraction unit, and to extract the centerline of the blood vessel segment;

the time difference unit is configured to receive any two frames of the two-dimensional coronary artery angiogram images sent by the image reading unit, and to determine a difference in time taken for a contrast agent flowing through the blood vessel segment in the two frames of two-dimensional coronary artery angiogram image with the difference being At;

the centerline difference unit is configured to receive the centerline of a sub-segment of the blood vessel segment flowed through by the contrast agent in the two frames of two-dimensional coronary artery angiogram image sent by the centerline extraction unit, and to determine a difference in centerline length of the sub-segment of the blood vessel segment through which the contrast agent flows in the two frames of two-dimensional coronary artery angiogram image with the difference being ΔL;

the blood flow velocity acquisition unit comprises a blood flow velocity calculation module and a diastolic blood flow velocity calculation module, the blood flow velocity calculation module being respectively connected to the time difference unit and the centerline difference unit, the diastolic blood flow velocity calculation module being connected with the blood flow velocity calculation module;

the blood flow velocity calculation module is configured to receive the ΔL and the Δt sent by the time difference unit and the centerline difference unit, and to solve the blood flow velocity according to the ratio of ΔL to Δt ;

the diastolic blood flow velocity calculation module is configured to receive the blood flow velocity sent by the blood flow velocity calculation module, and to select a maximum value of the blood flow velocity as a blood flow velocity during a diastolic phase;

the physiological parameter acquisition unit is configured to receive the two-dimensional coronary artery angiogram images of the image reading unit, to acquire a physiological parameter of a patient and image shooting angles, and to transmit the physiological parameter and image shooting angles to the unit of index for microcirculatory resistance during diastolic phase.

Optionally, the above apparatus for adjusting blood flow velocity in maximum hyperemia state based on index of microcirculatory resistance further comprises: a blood vessel skeleton extraction unit and a three-dimensional blood vessel reconstruction unit, both connected to the image reading unit, a contour line extraction unit connected to the blood vessel skeleton extraction unit, the three-dimensional blood vessel reconstruction unit being connected with the physiological parameter acquisition unit, the centerline extraction unit and the contour line extraction unit;

the blood vessel skeleton extraction unit is configured to receive the two-dimensional coronary artery angiogram images sent by the image reading unit, and to extract a blood vessel skeleton in the images;

the contour line extraction unit is configured to receive the blood vessel skeleton of the blood vessel skeleton extraction unit, and to extract a contour line of the blood vessel segment of interest according to the blood vessel skeleton;

the three-dimensional blood vessel reconstruction unit is configured to receive the contour line, the image shooting angles and the centerline sent by the contour line extraction unit, the physiological parameter acquisition unit and the centerline extraction unit, and to receive the two-dimensional coronary artery angiogram images sent by the image reading unit in order to synthesize a three-dimensional blood vessel model by projecting at least two body positions' two-dimensional coronary angiogram images which have been extracted centerline and contour line of the blood vessel onto a three-dimensional plane according to the geometric structure information of the blood vessel segment;

the centerline extraction unit is configured to re-extract the centerline of the blood vessel segment from the three-dimensional blood vessel model of the three-dimensional blood vessel reconstruction unit, and to re-acquire the length of the centerline.

In a fourth aspect, the present disclosure provides a coronary artery analysis system, comprising: the apparatus for adjusting blood flow velocity in maximum hyperemia state based on index for microcirculatory resistance according to any one of the above.

In a fifth aspect, the present disclosure provides a computer storage medium having stored thereon a computer program to be executed by a processor, the above method for adjusting blood flow velocity in maximum hyperemia state based on index for microcirculatory resistance is implemented when the computer program is executed by the processor.

The beneficial effects brought about by the solutions provided by the embodiments of the present disclosure comprise at least the following:

According to the present disclosure, an index for microcirculatory resistance iFMR during a diastolic phase is acquired according to a blood flow velocity v, an aortic pressure waveform, and an physiological parameter; then an adjustment parameter is obtained by comparing iFMR with K, where the adjustment parameter varies for different values of iFMR, further, a differentiated parameter is obtained according to individualized differences, placing a solid foundation for the accuracy of a blood vessel calculation parameter, then, a corrected blood flow velocity in the maximum hyperemia state is obtained by a product of the adjustment parameter and the blood flow velocity in the maximum hyperemia state, allowing for more targeted. individualized and more accurate measurement results.

### BRIEF DESCRIPTION OF DRAWINGS

The drawings illustrated here are used to provide a further understanding of the present disclosure and constitute a part of the present disclosure. The exemplary embodiments and the descriptions thereof are used to explain the present disclosure, and do not constitute an improper limitation on the present disclosure. In the drawings:

FIG. 1 is a flowchart of a method for adjusting blood flow velocity in maximum hyperemia state based on index for microcirculatory resistance of the present disclosure;

FIG. 2 is a flowchart of S100 of the present disclosure;

FIG. 3 is a flowchart of S120 of the present disclosure;

FIG. 4 is a flowchart of S130 of the present disclosure;

FIG. 5 is a flowchart of an embodiment of S140 of the present disclosure;

FIG. 6 is a flowchart of another embodiment of S140 of the present disclosure;

FIG. 7 is a structural block diagram of an embodiment of an apparatus for adjusting blood flow velocity in maximum hyperemia state based on index for microcirculatory resistance of the present disclosure;

FIG. 8 is a structural block diagram of another embodiment of an apparatus for adjusting blood flow velocity in maximum hyperemia state based on index for microcirculatory resistance of the present disclosure;

Reference numerals are explained below:

blood flow velocity acquisition unit 1, blood flow velocity calculation module 101, diastolic blood flow velocity calculation module 102, aortic pressure waveform acquisition unit 2, physiological parameter acquisition unit 3, unit of index for microcirculatory resistance during diastolic phase 4, adjustment parameter unit 5, image reading unit 6, blood vessel segment extraction unit 7, centerline extraction unit 8, time difference unit 9, centerline difference unit 10, blood vessel skeleton extraction unit 11, three-dimensional blood vessel reconstruction unit 12, contour line extraction unit 13, flow velocity correction unit 14, unit of flow velocity in maximum hyperemia state 15.

### DETAILED DESCRIPTION OF PREFERRED EMBODIMENTS

In order to make objects, technical solutions and advantages of the present disclosure clearer, the technical solutions of the present disclosure will be clearly and completely described below with reference to the specific embodiments and corresponding drawings. It is apparent that the described embodiments are merely part of the embodiments of the present disclosure rather than all of them. Based on the embodiments in the present disclosure, without making creative work, all the other embodiments obtained by a person skilled in the art will fall into the protection scope of the present disclosure.

Hereinafter, a number of embodiments of the present disclosure will be disclosed with drawings. For clear illustration, many practical details will be described in the following description. However, it should be understood that the present disclosure should not be limited by these practical details. In other words, in some embodiments of the present disclosure, these practical details are unnecessary. In addition, in order to simplify the drawings, some conventionally used structures and components will be shown in simple schematic ways in the drawings.

Due to different vital signs of different populations, evaluation standards for normal values are slightly different. For example, the myocardial microcirculation function of the elderly is relatively poor, and the blood flow velocity is generally lower than that of the young. If the industry general evaluation standard is used, the blood flow velocity used will be higher than the actual value. The higher blood flow velocity will further affect the coronary artery evaluation parameters, such as: fractional flow reserve FFR, fractional flow reserve during a diastolic phase iFR, and index for microcirculatory resistance iFMR during the diastolic phase.

Therefore, at this stage, how to obtain an adjustment parameter according to individual differences, for improving the accuracy of a blood vessel calculation parameter, such as a blood flow velocity, has become an urgent problem in the field of coronary artery technology.

### Embodiment 1

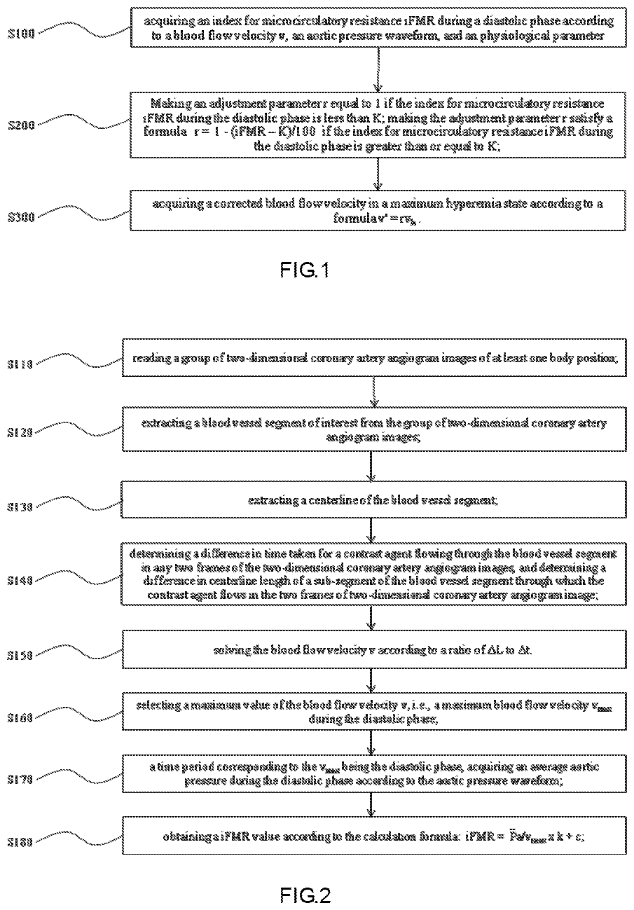

In order to solve the above problem, as shown in FIG. 1, the present disclosure provides a method for adjusting blood flow velocity in maximum hyperemia state based on index for microcirculatory resistance, comprising:

S100, acquiring an index for microcirculatory resistance iFMR during a diastolic phase according to a blood flow velocity v, an aortic pressure waveform, and an physiological parameter;

S00, making an adjustment parameter r equal to 1 if the index for microcirculatory resistance iFMR during the diastolic phase is less than K; making the adjustment parameter r satisfy a formula

$$r = 1 - \frac{iFMR - K}{100}$$

if the index for microcirculatory resistance iFMR during the diastolic phase is greater than or equal to K, wherein K is a positive number less than 100;

S300, acquiring a corrected blood flow velocity in a maximum hyperemia state according to a formula v′=rvₕ;

wherein v′ represents the corrected blood flow velocity in the maximum hyperemia state, and vₕ represents a blood flow velocity in the maximum hyperemia state.

In an embodiment of the present disclosure, vₕ=zv̅+x;

wherein vₕ represents the blood flow velocity in the maximum hyperemia state, v̅ represents an average blood flow velocity in a heartbeat cycle area, z is a constant in the range of 1 to 3, and x is a constant in the range of 50 to 300; K=50.

According to the present disclosure, an index for microcirculatory resistance iFMR during a diastolic phase is acquired according to a blood flow velocity v, an aortic pressure waveform, and an physiological parameter; then an adjustment parameter is obtained by comparing iFMR with K, where the adjustment parameter varies for different values of iFMR, further, a differentiated parameter is obtained according to individualized differences, placing a solid foundation for the accuracy of a blood vessel calculation parameter, then, a corrected blood flow velocity in the maximum hyperemia state is obtained by a product of the adjustment parameter and the blood flow velocity in the maximum hyperemia state, allowing for more targeted, individualized and more accurate measurement results.

### Embodiment 2

The present disclosure provides a method for adjusting blood flow velocity in maximum hyperemia state based on index for microcirculatory resistance, comprising:

S100, acquiring an index for microcirculatory resistance iFMR during a diastolic phase according to a blood flow velocity v, an aortic pressure waveform, and an physiological parameter; wherein

1) as shown in FIG. 2, when acquiring the blood flow velocity through a two-dimensional angiogram image, a manner for acquiring the blood flow velocity v comprises:

S110, reading a group of two-dimensional coronary artery angiogram images of at least one body position;

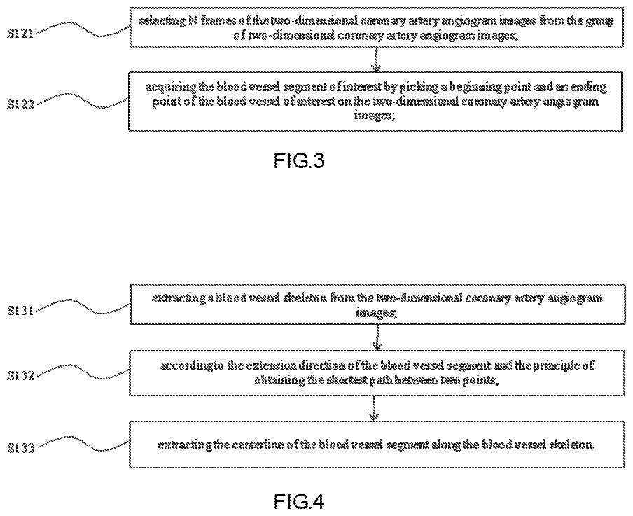

S120, extracting a blood vessel segment of interest from the group of two-dimensional coronary artery angiogram images, for which a specific manner is shown in FIG. 3, comprising:

S121, selecting N frames of the two-dimensional coronary artery angiogram images from the group of two-dimensional coronary artery angiogram images:

S122, acquiring the blood vessel segment of interest by picking a beginning point and an ending point of the blood vessel of interest on the two-dimensional coronary artery angiogram images;

S130, extracting a centerline of the blood vessel segment, for which a specific manner is shown in FIG. 4, comprising:

S131, extracting a blood vessel skeleton from the two-dimensional coronary artery angiogram images:

S132, according to the extension direction of the blood vessel segment and the principle of obtaining the shortest path between two points:

S133, extracting the centerline of the blood vessel segment along the blood vessel' skeleton.

S140, determining a difference in time taken for a contrast agent flowing through the blood vessel segment in any two frames of the two-dimensional' coronary artery angiogram images with the difference being Δt , and determining a difference in centerline length of a sub-segment of the blood vessel segment through which the contrast agent flows in the two frames of two-dimensional coronary artery angiogram image with the difference being ΔL;

S150, solving the blood flow velocity according to a ratio of Δl to Δt.

2) A manner for acquiring an aortic pressure waveform comprises:

acquiring, in real time, an aortic pressure Pₐⱼ with an invasive blood pressure sensor or non-invasive blood pressure instrument, where j is a positive integer greater than or equal to 1, and then generating an aortic pressure waveform according to time;

3) A manner for acquiring the index for microcirculatory resistance iFMR during the diastolic phase comprises:

S160, selecting a maximum value of the blood flow velocity v obtained by solving in S150, i.e., a maximum blood flow velocity vₘₐₓ during the diastolic phase;

S170, a time period corresponding to the vₘₐₓ being the diastolic phase, acquiring an average aortic pressure during the diastolic phase according to the aortic pressure waveform, namely:

$$\overline{{P}_{a}} = \frac{{\sum }_{1}^{j} ( {P}_{a 1} + {P}_{a 2} … {P}_{aj} )}{j} ;$$

S180, obtaining a iFMR value according to the calculation formula:

iFMR=P̅ₐ̅/vₘₐₓ×k+c;

wherein, P̅ₐ̅ represents the average aortic pressure during the diastolic phase; , and represent aortic pressures corresponding to a first point, a second point, and a j-th point within the diastolic phase on the aortic pressure waveform, respectively, and j represents the number of pressure points contained in the aortic pressure waveform during the diastolic phase, vₕ represents the blood flow velocity in the maximum hyperemia state obtained by selecting a maximum value from all blood flow velocities v, preferably by selecting a maximum value of blood flow velocity through a recursive algorithm or a bubbling algorithm; k and c represent the influence parameters k=1˜3, c=0˜10;

S200, making an adjustment parameter r equal to 1 if the index for microcirculatory resistance iFMR during the diastolic phase is less than K; making the adjustment parameter r satisfy a formula

$$r = 1 - \frac{iFMR - K}{100}$$

if the index for microcirculatory resistance iFMR during the diastolic phase is greater than or equal to K,

wherein K is a positive number less than 100; preferably, K=50.

All the contents of acquiring coronary artery blood vessel evaluation parameter based on the physiological parameter are within the scope of protection of the present disclosure. After a large number of experimental verifications, histories of having hypertension and diabetes and gender all have impacts on the accuracy of calculating a coronary artery blood vessel evaluation parameter. Therefore, in an embodiment of the present disclosure_{;} an influence parameter kin S180 is calculated by the formula: k=a×b, where a represents a characteristic value of diabetes, b represents a characteristic value of hypertension, and c represents the gender. If a patient does not suffer from diabetes, then 0.5≤a≤1, preferably, a=1; if the patient suffers from diabetes, then 1<a≤2, preferably, a=2; if the patient's blood pressure value is greater than or equal to 90 mmHg, then 1<b≤1.5, preferably, b=1.5; if the patient's blood pressure value is less than 90 mmHg, then 0.5≤b≤1, preferably, b=1; if the patient is male, then c=0; if the patient is female, then c=3˜10, preferably, c=5.

In an embodiment of the present disclosure, S140 comprises two acquisition methods. As shown in FIG. 5, method(1) comprises:

S141I, taking the coronary angiogram image when the contrast agent flows to the inlet of the coronary artery, that is, the beginning point of the blood vessel segment as a first frame of image, and taking the coronary angiogram image when the contrast agent flows to the ending point of the blood vessel segment as a N-th frame of image;

S142I, solving the time difference and centerline length difference of the N-th frame of image and a (N−1)th frame, . . . , a (N−b)th frame, . . . , a (N−a)th frame, . . . , the first frame of image, successively, with the time differences being Δt₁, . . . , Δt_{b}, . . . , Δtₐ, . . . , Δt_{N-1}, respectively; the centerline length differences being ΔL₁, . . . , ΔL_{b}, . . . , ΔLₐ, . . . , respectively;

S143I, according to v=ΔL/Δt, obtaining the blood flow velocity from the N-th frame of image to the (N−1)th frame, . . . , the (N−b)th frame, . . . , the (N−a)th frame, . . . , the first frame of image, respectively, wherein v represents the blood flow velocity, with the blood flow velocity being v₁. . . . , v_{b}, . . . , vₐ, . . . , V_{N-1}, respectively.

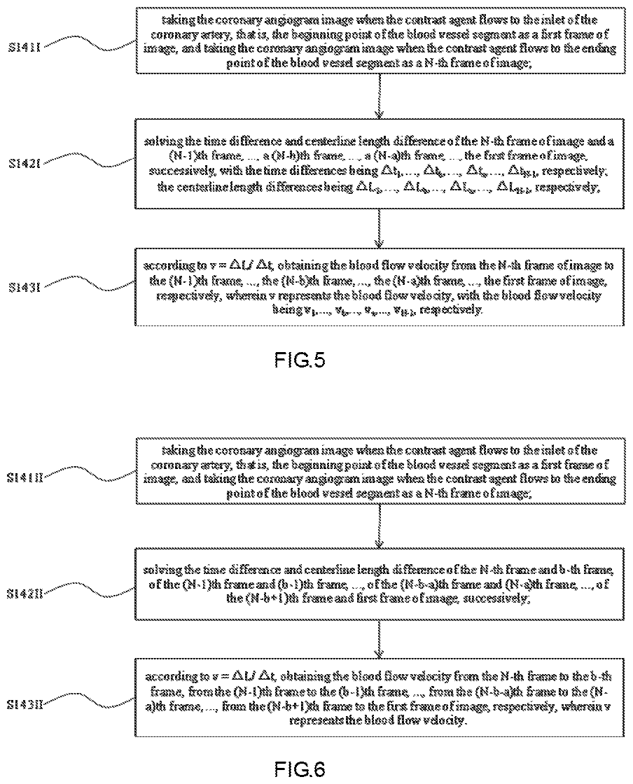

In an embodiment of the present disclosure, 5140 comprises two acquisition methods. As shown in FIG. 6, method(2) comprises:

S141II, taking the coronary angiogram image when the contrast agent flows to the inlet of the coronary artery, that is, the beginning point of the blood vessel segment as a first frame of image, and taking the coronary angiogram image when the contrast agent flows to the ending point of the blood vessel segment as a N-th frame of image;

S142II, solving the time difference and centerline length difference of the N-th frame and b-th frame, of the (N−1)th frame and (b−1)th frame, . . . , of the (N−b−a)th frame and (N−a)th frame, . . . , of the (N−b+1)th frame and first frame of image. successively;

S143II, according to v=ΔL/Δt, obtaining the blood flow velocity from the N-th frame to the b-th frame, from the (N−1)th frame to the (b−1)th frame, . . . , from the (N−b−a)th frame to the (N−a)th frame, . . . , from the (N−b+1)th frame to the first frame of image, respectively, wherein v represents the blood flow velocity.

### Embodiment 3

In an embodiment of the present disclosure, a manner for acquiring a blood flow velocity by three-dimensional modeling in S100 comprises:

Step A, reading a group of two-dimensional coronary artery angiogram images of at least two body positions;

Step B, extracting a blood vessel segment of interest from the groups of two-dimensional coronary artery angiogram images;

Step C, acquiring geometric structure information of the blood vessel segment and extracting a centerline of the blood vessel segment;

Step D, performing graphics processing on the blood vessel segment of interest;

Step E, extracting a blood vessel contour line of the blood vessel segment;

Step F, according to the geometric structure information of the blood vessel segment, synthesizing a three-dimensional blood vessel model by projecting the at least two body positions' two-dimensional coronary angiogram images which have been extracted centerline and contour line of the blood vessel onto a three-dimensional plane;

Step G, determining a difference in time taken for a contrast agent flowing through the blood vessel segment in any two frames of the two-dimensional coronary artery angiogram images with the difference being Δt; according to the three-dimensional blood vessel model, acquiring a centerline of the three-dimensional blood vessel model, correcting the centerline extracted from the two-dimensional coronary angiogram images, and determining a difference in the corrected centerline length of a sub-segment of the blood vessel segment through which the contrast agent flows in the two frames of two-dimensional coronary artery angiogram image with the difference being ΔL′; solving the blood flow velocity v according to the ratio of the ΔL′ to the Δt.

Δt=m×fps, since each group of two-dimensional coronary artery angiogram images contains multiple frames of two-dimensional coronary artery angiogram images played consecutively, m represents a difference in frame number between two frames of two-dimensional angiogram image selected from each group of two-dimensional coronary artery angiogram images, and fps represents an interval time for switching between two adjacent frames of the image, preferably, fps= 1/15 second.

### Embodiment 4

The present disclosure provides a method for acquiring blood flow velocity in maximal hyperemia state based on physiological parameter, comprising:

the above method for adjusting blood flow velocity in maximum hyperemia state based on index for microcirculatory resistance;

according to the formula v′=rvₕ;

$${v}_{h} = z \overline{v} + x ;$$

$$r = 1 - \frac{iFMR - K}{100} ; iFMR = \overline{{P}_{a}} / {v}_{max} \times k + c .$$

wherein v′ represents a blood flow velocity in the maximum hyperemia state which has been adjusted by a physiological parameter, vₕ represents a blood flow velocity in the maximum hyperemia state, v̅ represents an average blood flow velocity in a heartbeat cycle area, z is a constant in the range of 1 to 3, and x is a constant in the range of 50 to 300.

An embodiment of the present disclosure provides a method for acquiring coronary artery blood vessel evaluation parameter based on physiological parameter, comprising the above method for acquiring blood flow velocity in maximal hyperemia state based on physiological parameter. A coronary artery blood vessel evaluation parameter comprises: fractional flow reserve FFR. index for microcirculatory resistance IMR, index for microcirculatory resistance iFMR during a diastolic phase, fractional flow reserve iFR during the diastolic phase and the like.

### Embodiment 5

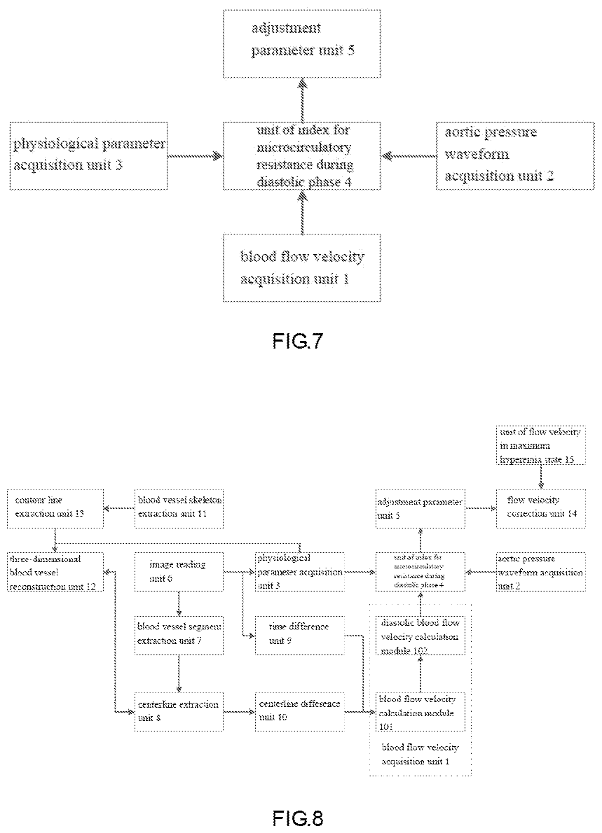

As shown in FIG. 7 the present disclosure provides an apparatus for adjusting blood flow velocity in maximum hyperemia state based on index for microcirculatory resistance, which is used for the above method for adjusting blood flow velocity in maximum hyperemia state based on index for microcirculatory resistance, comprising: a blood flow velocity acquisition unit 1, an aortic pressure waveform acquisition unit 2, a physiological parameter acquisition unit 3, a unit of index for microcirculatory resistance during diastolic phase 4 and an adjustment parameter unit 5. The unit of index for microcirculatory resistance during diastolic phase 4 is connected with the blood flow velocity acquisition unit 1, the aortic pressure waveform acquisition unit 2 and the physiological parameter acquisition unit 3. The blood flow velocity acquisition unit 1 is configured to acquire a blood flow velocity v. The aortic pressure waveform acquisition unit 2 is configured to acquire, in real time, an aortic pressure waveform changing over time. The physiological parameter acquisition unit 3 is configured to acquire physiological parameters of a patient, comprising gender and disease history. The unit of index for microcirculatory resistance during diastolic phase 4 is configured to receive the blood flow velocity v, the aortic pressure waveform, and the physiological parameters sent by the blood flow velocity acquisition unit 1, the aortic pressure waveform acquisition unit 2, and the physiological parameter acquisition unit 3, and then to obtain an index for microcirculatory resistance iFMR during a diastolic phase according to the blood flow velocity v, the aortic pressure waveform, and the physiological parameters. The adjustment parameter unit 5 is configured to receive iFMR value of the unit of index for microcirculatory resistance during diastolic phase 4. An adjustment parameter r is equal to 1 if the index for microcirculatory resistance during the diastolic phase iFMR<K; the adjustment parameter r satisfies a formula

$$r = 1 - \frac{iFMR - K}{100}$$

if the index for microcirculatory resistance during the diastolic phase iFMR≥K; where K is a positive number less than 100.

As shown in FIG. 8, an embodiment of the present disclosure further comprises: a flow velocity correction unit 14 connected to the adjustment parameter unit 5, a unit of flow velocity in maximum hyperemia state 15 connected to the flow velocity correction unit 14. The unit of flow velocity in maximum hyperemia state 15 is configured to calculate a blood flow velocity in a maximum hyperemia state according to vₕ=zv̅+x; vₕ represents the blood flow velocity in the maximum hyperemia state, v̅ represents an average blood flow velocity in a heartbeat cycle area, z is a constant n the range of 1 to 3, and x is a constant in the range of 50 to 300.

As shown in FIG. 8, an embodiment of the present application further comprises: an image reading unit 6, a blood vessel segment extraction unit 7, and a centerline extraction unit 8 connected in sequence, a time difference unit 9 and the physiological parameter acquisition unit 3 connected to the image reading unit 6, and the blood flow velocity acquisition unit 1 respectively connected with the time difference unit 9 and a centerline difference unit 10. The centerline difference unit 10 is connected with the centerline extraction unit 8. The image reading unit 6 is configured to read a group of two-dimensional coronary artery angiogram image of at least one body position. The blood vessel segment extraction unit 7 is configured to receive two-dimensional coronary artery angiogram images sent by the image reading unit 6, and to extract a blood vessel segment of interest in the images. The centerline extraction unit 8 is configured to receive the blood vessel segment sent by the blood vessel segment extraction unit 7, and to extract the centerline of the blood vessel segment. The time difference unit 9 is configured to receive any two frames of the two-dimensional coronary artery angiogram images sent by the image reading unit 6, and to determine a difference in time taken for a contrast agent flowing through the blood vessel segment in the two frames of two-dimensional coronary artery angiogram image with the difference being Δt. The centerline difference unit 10 is configured to receive the centerline of a sub-segment of the blood vessel segment flowed through by the contrast agent in the two frames of two-dimensional coronary artery angiogram image sent by the centerline extraction unit, and to determine a difference in centerline length of the sub-segment of the blood vessel segment through which the contrast agent flows in the two frames of two-dimensional coronary artery angiogram image with the difference being ΔL. The blood flow velocity acquisition unit 1 comprises a blood flow velocity calculation module 101 and a diastolic blood flow velocity calculation module 102. The blood flow velocity calculation module 101 is respectively connected to the time difference unit 9 and the centerline difference unit 10. The diastolic blood flow velocity calculation module 102 is connected with the blood flow velocity calculation module 101. The blood flow velocity calculation module 101 is configured to receive the ΔL and the Δt sent by the time difference unit 9 and the centerline difference unit 10, and to solve the blood flow velocity according to the ratio of ΔL to Δt. The diastolic blood flow velocity calculation module 102 is configured to receive the blood flow velocity sent by the blood flow velocity calculation module 101, and to select a maximum value of the blood flow velocity as a blood flow velocity during a diastolic phase. The physiological parameter acquisition unit 3 is configured to receive the two-dimensional coronary artery angiogram images of the image reading unit 6, to acquire a physiological parameter of a patient, image shooting angles and imaging distance, and to transmit the physiological parameter, image shooting angles and imaging distance to the unit of index for microcirculatory resistance during diastolic phase 4.

The above imaging distance may be understood as: when synthesizing a three-dimensional model by two plane images, as long as the distance between the object and the imaging plane, the image shooting angle, and the two two-dimensional plane images are known, the three-dimensional model can be generated through the principle of three-dimensional imaging.

In an embodiment of the present application, the apparatus further comprises: a blood vessel skeleton extraction unit 11 and a three-dimensional blood vessel reconstruction unit 12, both connected to the image reading unit 6, a contour line extraction unit 13 connected to the blood vessel skeleton extraction unit 11. The three-dimensional blood vessel reconstruction unit 12 is connected with the physiological parameter acquisition unit 3, the centerline extraction unit 8 and the contour line extraction unit 13. The blood vessel skeleton extraction unit 11 is configured to receive the two-dimensional coronary artery angiogram images sent by the image reading unit 6, and to extract a blood vessel skeleton in the images. The contour line extraction unit 13 is configured to receive the blood vessel skeleton of the blood vessel skeleton extraction unit 11, and to extract a contour line of the blood vessel segment of interest according to the blood vessel skeleton. The three-dimensional blood vessel reconstruction unit 12 is configured to receive the contour line, the image shooting angles and the centerline sent by the contour line extraction unit 13, the physiological parameter acquisition unit 3 and the centerline extraction unit 8, and to receive the two-dimensional coronary artery angiogram images sent by the image reading unit 6 in order to synthesize a three-dimensional blood vessel model by projecting at least two body positions' two-dimensional coronary angiogram images which have been extracted centerline and contour line of the blood vessel onto a three-dimensional plane according to the geometric structure information of the blood vessel segment. The centerline extraction unit 8 is configured to re-extract the centerline of the blood vessel segment from the three-dimensional blood vessel model of the three-dimensional blood vessel reconstruction unit 12, and to re-acquire the length of the centerline.

The present disclosure provides a coronary artery analysis system, which comprises the apparatus for adjusting blood flow velocity in maximum hyperemia state based on index for microcirculatory resistance according to any one of the above.

The present disclosure provides a computer storage medium having stored thereon a computer program to be executed by a processor, wherein the above method for adjusting blood flow velocity in maximum hyperemia state based on index for microcirculatory resistance is implemented when the computer program is executed by the processor.

A person skilled in the art knows that various aspects of the present disclosure can be implemented as a system, a method, or a computer program product. Therefore, each aspect of the present disclosure can be specifically implemented in the following forms, namely: complete hardware implementation, complete software implementation (including firmware, resident software, microcode, etc.), or a combination of hardware and software implementations, which here can be collectively referred to as “circuit”, “module” or “system”. In addition, in some embodiments, various aspects of the present disclosure may also be implemented in the form of a computer program product in one or more computer-readable media, and the computer-readable medium contains computer-readable program code. Implementation of a method and/or a system of embodiments of the present disclosure may involve performing or completing selected tasks manually, automatically, or a combination thereof.

For example, hardware for performing selected tasks according to the embodiment(s) of the present disclosure may be implemented as a chip or a circuit. As software, selected tasks according to the embodiment(s) of the present disclosure can be implemented as a plurality of software instructions executed by a computer using any suitable operating system. In the exemplary embodiment(s) of the present disclosure, a data processor performs one or more tasks according to the exemplary embodiment(s) of a method and/or system as described herein, such as a computing platform for executing multiple instructions. Optionally, the data processor comprises a volatile memory for storing instructions and/or data, and/or a non-volatile memory for storing instructions and/or data, for example, a magnetic hard disk and/or movable medium. Optionally, a network connection is also provided. Optionally, a display and/or user input device, such as a keyboard or mouse, are/is also provided.

Any combination of one or more computer readable media can be utilized. The computer-readable medium may be a computer-readable signal medium or a computer-readable storage medium. The computer-readable storage medium may be, for example, but not limited to, an electrical, magnetic, optical, electromagnetic, infrared, or semiconductor system, apparatus, or device, or any combination of the above. More specific examples (non-exhaustive list) of computer-readable storage media would include the following:

Electrical connection with one or more wires, portable computer disk, hard disk, random access memory (RAM), read only memory (ROM), erasable programmable read only memory (EPROM or flash memory), optical fiber, portable compact disk read only memory (CD-ROM), optical storage device, magnetic storage device, or any suitable combination of the above. In this document, the computer-readable storage medium can be any tangible medium that contains or stores a program, and the program can be used by or in combination with an instruction execution system, apparatus, or device.

The computer-readable signal medium may include a data signal propagated in baseband or as a part of a carrier wave, which carries computer-readable program code. This data signal for propagation can take many forms, including but not limited to electromagnetic signals, optical signals, or any suitable combination of the above. The computer-readable signal medium may also be any computer-readable medium other than the computer-readable storage medium. The computer-readable medium can send, propagate, or transmit a program for use by or in combination with the instruction execution system, apparatus, or device.

The program code contained in the computer-readable medium can be transmitted by any suitable medium, including, but not limited to, wireless, wired, optical cable, RF, etc., or any suitable combination of the above.

For example, any combination of one or more programming languages can be used to write computer program codes for performing operations for various aspects of the present disclosure, including object-oriented programming languages such as Java, Smalltalk, C++, and conventional process programming languages, such as “C” programming language or similar programming language. The program code can be executed entirely on a user's computer, partly on a user's computer, executed as an independent software package, partly on a user's computer and partly on a remote computer, or entirely on a remote computer or server. In the case of a remote computer, the remote computer can be connected to a user's computer through any kind of network including a local area network (LAN) or a wide area network (WAN), or it can be connected to an external computer (for example. connected through Internet provided by an Internet service provider).

It should be understood that each block of the flowcharts and/or block diagrams and combinations of blocks in the flowcharts and/or block diagrams can be implemented by computer program instructions. These computer program instructions can be provided to the processor of general-purpose computers, special-purpose computers, or other programmable data processing devices to produce a machine. which produces a device that implements the functions/actions specified in one or more blocks in the flowcharts and/or block diagrams when these computer program instructions are executed by the processor of the computer or other programmable data processing devices.

It is also possible to store these computer program instructions in a computer-readable medium. These instructions make computers, other programmable data processing devices, or other devices work in a specific manner, so that the instructions stored in the computer-readable medium generate an article of manufacture comprising instructions for implementation of the functions/actions specified in one or more blocks in the flowcharts and/or block diagrams.

Computer program instructions can also be loaded onto a computer (for example, a coronary artery analysis system) or other programmable data processing equipment to facilitate a series of operation steps to be performed on the computer, other programmable data processing apparatus or other apparatus to produce a computer-implemented process, which enable instructions executed on a computer, other programmable device, or other apparatus to provide a process for implementing the functions/actions specified in the flowcharts and/or one or more block diagrams.

The above specific examples of the present disclosure further describe the purpose, technical solutions and beneficial effects of the present disclosure in detail. It should be understood that the above are only specific embodiments of the present disclosure and are not intended to limit the present disclosure. Within the spirit and principle of the present disclosure, any modification, equivalent replacement, improvement, etc. shall be included in the protection scope of the present disclosure.

What is claimed is:

1. A method for adjusting blood flow velocity in maximum hyperemia state based on index for microcirculatory resistance, comprising:
acquiring an index for microcirculatory resistance iFMR during a diastolic phase according to a blood flow velocity v, an aortic pressure waveform, and an physiological parameter:
making an adjustment parameter r equal to 1 if the index for microcirculatory resistance iFMR during the diastolic phase is less than K; making the adjustment parameter r satisfy a formula
$$r = 1 - \frac{iFMR - K}{100}$$
if the index for microcirculatory resistance iFMR during the diastolic phase is greater than or equal to K, wherein K is a positive number less than 100;
acquiring a corrected blood flow velocity in a maximum hyperemia state according to a formula v′=rvₕ;
wherein v′ represents the corrected blood flow velocity in the maximum hyperemia state, and vₕ represents a blood flow velocity in the maximum hyperemia state.

2. The method for adjusting blood flow velocity in maximum hyperemia state based on index for microcirculatory resistance according to claim 1, wherein,
vₕ=zv̅+x
wherein vₕ represents the blood flow velocity in the maximum hyperemia state, v̅ represents an average blood flow velocity in a heartbeat cycle area, z is a constant in the range of 1 to 3, and x is a constant in the range of 50 to 300; K=50.

3. The method for adjusting blood flow velocity in maximum hyperemia state based on index for microcirculatory resistance according to claim 1, wherein a manner for acquiring an index for microcirculatory resistance iFMR during a diastolic phase according to a blood flow velocity v, an aortic pressure waveform, and an physiological parameter comprises:
selecting a maximum value of the blood flow velocity v, i.e., a maximum blood flow velocity vₘₐₓ during the diastolic phase;
a time period corresponding to the vₘₐₓ being the diastolic phase, acquiring an average aortic pressure during the diastolic phase according to the aortic pressure waveform;
$$iFMR = \overline{{P}_{a}} / {v}_{max} \times k + c ;$$
$$\overline{{P}_{a}} = \frac{{\sum }_{1}^{j} ( {P}_{a 1} + {P}_{a 2} … {P}_{aj} )}{j} ;$$
wherein, P̅ₐ̅ represents the average aortic pressure during the diastolic phase: Pₐ₁, Pₐ₂, and Pₐⱼ represent aortic pressures corresponding to a first point, a second point, and a j-th point within the diastolic phase on the aortic pressure waveform, respectively, and j represents the number of pressure points contained in the aortic pressure waveform during the diastolic phase, vₕ represents the blood flow velocity in the maximum hyperemia state obtained by selecting a maximum value from all blood flow velocities v; k and c represent the influence parameters k=1—3, c=0—10.

4. The method for adjusting blood flow velocity in maximum hyperemia state based on index for microcirculatory resistance according to claim 3, wherein the influence parameter k=a×b, wherein a represents a characteristic value of diabetes, b represents a characteristic value of hypertension, and c represents gender.

5. The method for adjusting blood flow velocity in maximum hyperemia state based on index for microcirculatory resistance according to claim 4, wherein if a patient does not suffer from diabetes, then 0.5≤a≤1; if the patient suffers from diabetes, then 1<a≤2;
if the patient's blood pressure is greater than or equal to 90 mmHg, then 1<b≤1.5; if the patient's blood pressure is less than 90 mmHg, then 0.5≤b≤1;
if the patient is male, then c=0; if the patient is female, then c=3˜10.

6. The method for adjusting blood flow velocity in maximum hyperemia state based on index for microcirculatory resistance according to claim 5, wherein if the patient does not suffer from diabetes, then a=1; If the patient suffers from diabetes, then a=2;
if the patients blood pressure is greater than or equal to 90 mmHg, then b=1.5; if the patient's blood pressure is less than 90 mmHg, then b=1;
if the patient is male, c=0; if the patient is female, c=5.

7. The method for adjusting blood flow velocity in maximum hyperemia state based on index for microcirculatory resistance according to claim 1, wherein a manner for acquiring a blood flow velocity comprises:
reading a group of two-dimensional coronary artery angiogram images of at least one body position;
extracting a blood vessel segment of interest from the group of two-dimensional coronary artery angiogram images;
extracting a centerline of the blood vessel segment;
determining a difference in time taken for a contrast agent flowing through the blood vessel segment in any two frames of the two-dimensional coronary artery angiogram images with the difference being Δt , and determining a difference in centerline length of a sub-segment of the blood vessel segment through which the contrast agent flows in the two frames of two-dimensional coronary artery angiogram image with the difference being ΔL;
solving the blood flow velocity according to a ratio of ΔL to Δt.

8. The method for adjusting blood flow velocity in maximum hyperemia state based on index for microcirculatory resistance according to claim 7, wherein a manner for extracting a blood vessel segment of interest from the group of two-dimensional coronary artery angiogram images comprises:
selecting N frames of the two-dimensional coronary artery angiogram images from the group of two-dimensional coronary artery angiogram images;
acquiring the blood vessel segment of interest by picking a beginning point and an ending point of the blood vessel of interest on the two-dimensional coronary artery angiogram images.

9. The method for adjusting blood flow velocity in maximum hyperemia state based on index for microcirculatory resistance according to claim 7, wherein a manner for extracting the centerline of the blood vessel segment comprises:
extracting a blood vessel skeleton from the two-dimensional coronary artery angiogram images;
according to an extension direction of the blood vessel segment and a principle of obtaining the shortest path between two points;
extracting the centerline of the blood vessel segment along the blood vessel skeleton.

10. The method for adjusting blood flow velocity in maximum hyperemia state based on index for microcirculatory resistance according to claim 7, wherein a manner for determining a difference in time taken for a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL, and solving the blood flow velocity according to the ratio of ΔL to Δt comprises:
taking the coronary angiogram image when the contrast agent flows to the inlet of the coronary artery, that is, the beginning point of the blood vessel segment as a first frame of image, and taking the coronary angiogram image when the contrast agent flows to the ending point of the blood vessel segment as a N-th frame of image;
solving the time difference and centerline length difference of the N-th frame of image and a (N−1)th frame, . . . , a (N−b)th frame, . . . , a (N−a)th frame, . . . , the first frame of image, successively, with the time differences being Δt₁, . . . , Δt_{b}, . . . , Δtₐ, . . . , Δt_{N-1}, respectively; the centerline length differences being ΔL₁, . . . , ΔL_{b}, . . . , ΔLₐ, . . . , ΔL_{N-1}, respectively;
according to v=ΔL/Δt, obtaining the blood flow velocity from the N-th frame of image to the (N−1)th frame, . . . , the (N−b)th frame, . . . , the (N−a)th frame, . . . , the first frame of image, respectively, wherein v represents the blood flow velocity, with the blood flow velocity being V₁, . . . , V_{b}, . . . , Vₐ, . . . , V_{N-1}, respectively.

11. The method for adjusting blood flow velocity in maximum hyperemia state based on index for microcirculatory resistance according to claim 7. wherein a manner for determining a difference in time taken a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL, and solving the blood flow velocity according to the ratio of ΔL to Δt comprises:
solving the time difference and centerline length difference of the N-th frame and b-th frame, of the (N−1)th frame and (b−1)th frame, . . . , of the (N−b−a)th frame and (N−a)th frame, . . . , of the (N−b+1)th frame and first frame of image, successively;
according to v=ΔL/Δt, obtaining the blood flow velocity from the N-th frame to the b-th frame, from the (N−1)th frame to the (b−1)th frame, . . . , from the (N−b−a)th frame to the (N−a)th frame, . . . , from the (N−b+1)th frame to the first frame of image, respectively, wherein v represents the blood flow velocity.

12. The method for adjusting blood flow velocity in maximum hyperemia state based on index for microcirculatory resistance according to claim 7, wherein, after the manner for extracting a centerline of the blood vessel segment, and before the manner for determining a difference in time taken for a contrast agent flowing through the blood vessel segment in any two frames of the two-dimensional coronary artery angiogram images with the difference being Δt, and determining a difference in centerline length of a sub-segment of the blood vessel segment through which the contrast agent flows in the two frames of two-dimensional coronary artery angiogram image with the difference being ΔL further comprises:
reading a group of two-dimensional coronary artery angiogram images of at least two body positions:
acquiring geometric structure information of the blood vessel segment;
performing graphics processing on the blood vessel segment of interest;
extracting a blood vessel contour line of the blood vessel segment;
according to the geometric structure information of the blood vessel segment, synthesizing a three-dimensional blood vessel model by projecting the at least two body position& two-dimensional coronary angiogram images which have been extracted centerline and contour line of the blood vessel onto a three-dimensional plane.

13. The method for adjusting blood flow velocity in maximum hyperemia state based on index for microcirculatory resistance according to claim 12, wherein a manner for solving the blood flow velocity according to the ratio of ΔL to Δt comprises:
according to the three-dimensional blood vessel model, acquiring a centerline of the three-dimensional blood vessel model, correcting the centerline extracted from the two-dimensional coronary angiogram images, and correcting the centerline difference ΔL to obtain ΔL′;
solving the blood flow velocity v according to the ratio of the ΔL′ to the Δt.

14. A method for acquiring coronary artery blood vessel evaluation parameter based on physiological parameter, comprising: the method for adjusting blood flow velocity in maximum hyperemia state based on index for microcirculatory resistance according to claim 13 .

15. An apparatus for adjusting blood flow velocity in maximum hyperemia state based on index for microcirculatory resistance, used for the method for adjusting blood flow velocity in maximum hyperemia state based on index for microcirculatory resistance according to claim 1, characterized by comprising: a blood flow velocity acquisition unit, an aortic pressure waveform acquisition unit, a physiological parameter acquisition unit, a unit of index for microcirculatory resistance during diastolic phase and an adjustment parameter unit; the unit of index for microcirculatory resistance during diastolic phase being connected with the blood flow velocity acquisition unit, the aortic pressure waveform acquisition unit and the physiological parameter acquisition unit;
the blood flow velocity acquisition unit being configured to acquire a blood flow velocity v;
the aortic pressure waveform acquisition unit being configured to acquire. in real time. an aortic pressure waveform changing over time;
the physiological parameter acquisition unit being configured to acquire physiological parameters of a patient, comprising gender and disease history;
the unit of index for microcirculatory resistance during diastolic phase being configured to receive the blood flow velocity v, the aortic pressure waveform, and the physiological parameters sent by the blood flow velocity acquisition unit, the aortic pressure waveform acquisition unit, and the physiological parameter acquisition unit, and then to obtain an index for microcirculatory resistance iFMR during a diastolic phase according to the blood flow velocity v, the aortic pressure waveform, and the physiological parameters;
the adjustment parameter unit being configured to receive iFMR value of the unit of index for microcirculatory resistance during diastolic phase; make an adjustment parameter r equal to 1 if the index for microcirculatory resistance during the diastolic phase iFMR<K; make the adjustment parameter r satisfy a formula
$$r = 1 - \frac{iFMR - K}{100}$$
it the index tor microcirculatory resistance during the diastolic phase iFMR≥K; where K is a positive number less than 100.

16. The apparatus for adjusting blood flow velocity in maximum hyperemia state based on index for microcirculatory resistance according to claim 15, further comprising: an image reading unit, a blood vessel segment extraction unit, and a centerline extraction unit connected in sequence, a time difference unit and the physiological parameter acquisition unit connected to the image reading unit, the blood flow velocity acquisition unit being connected with the time difference unit and a centerline difference unit, respectively; the centerline difference unit being connected with the centerline extraction unit;
the image reading unit being configured to read a group of two-dimensional coronary artery angiogram image of at least one body position;
the blood vessel segment extraction unit being configured to receive two-dimensional coronary artery angiogram images sent by the image reading unit, and to extract a blood vessel segment of interest in the images;
the centerline extraction unit being configured to receive the blood vessel segment sent by the blood vessel segment extraction unit, and to extract the centerline of the blood vessel segment;
the time difference unit being configured to receive any two frames of the two-dimensional coronary artery angiogram images sent by the image reading unit, and to determine a difference in time taken for a contrast agent flowing through the blood vessel segment in the two frames of two-dimensional coronary artery angiogram image with the difference being Δt;
the centerline difference unit being configured to receive the centerline of a sub-segment of the blood vessel segment flowed through by the contrast agent in the two frames of two-dimensional coronary artery angiogram image sent by the centerline extraction unit, and to determine a difference in centerline length of the sub-segment of the blood vessel segment through which the contrast agent flows in the two frames of two-dimensional coronary artery angiogram image with the difference being ΔL;
the blood flow velocity acquisition unit, comprising a blood flow velocity calculation module and a diastolic blood flow velocity calculation module, the blood flow velocity calculation module being respectively connected to the time difference unit and the centerline difference unit, the diastolic blood flow velocity calculation module being connected with the blood flow velocity calculation module;
the blood flow velocity calculation module being configured to receive the ΔL and the Δt sent by the time difference unit and the centerline difference unit, and to solve the blood flow velocity according to the ratio of ΔL to Δt;
the diastolic blood flow velocity calculation module being configured to receive the blood flow velocity sent by the blood flow velocity calculation module, and to select a maximum value of the blood flow velocity as a blood flow velocity during a diastolic phase;
the physiological parameter acquisition unit being configured to receive the two-dimensional coronary artery angiogram images of the image reading unit, to acquire a physiological parameter of a patient and image shooting angles, and to transmit the physiological parameter and image shooting angles to the unit of index for microcirculatory resistance during diastolic phase.

17. The apparatus for adjusting blood flow velocity in maximum hyperemia state based on index of microcirculatory resistance according to claim 16, further comprising: a blood vessel skeleton extraction unit and a three-dimensional blood vessel reconstruction unit, both connected to the image reading unit, a contour line extraction unit connected to the blood vessel skeleton extraction unit, the three-dimensional blood vessel reconstruction unit being connected with the physiological parameter acquisition unit. the centerline extraction unit and the contour line extraction unit:
the blood vessel skeleton extraction unit being configured to receive the two-dimensional coronary artery angiogram images sent by the image reading unit, and to extract a blood vessel skeleton in the images;
the contour line extraction unit being configured to receive the blood vessel skeleton of the blood vessel skeleton extraction unit, and to extract a contour line of the blood vessel segment of interest according to the blood vessel skeleton;
the three-dimensional blood vessel reconstruction unit being configured to receive the contour line, the image shooting angles and the centerline sent by the contour line extraction unit, the physiological parameter acquisition unit and the centerline extraction unit, and to receive the two-dimensional coronary artery angiogram images sent by the image reading unit in order to synthesize a three-dimensional blood vessel model by projecting the two-dimensional coronary angiogram images of at least two body positions with extracted centerline and contour line of the blood vessel onto a three-dimensional plane and according to the geometric structure information of the blood vessel segment;
the centerline extraction unit being configured to re-extract the centerline of the blood vessel segment from the three-dimensional blood vessel model of the three-dimensional blood vessel reconstruction unit, and to re-acquire the length of the centerline.

18. A coronary artery analysis system, comprising: the apparatus for adjusting blood flow velocity in maximum hyperemia state based on index for microcirculatory resistance according to claim 15.

19. A computer storage medium having stored thereon a computer program to be executed by a processor, wherein the method for adjusting blood flow velocity in maximum hyperemia state based on index for microcirculatory resistance according to claim 1 is implemented when the computer program is executed by the processor.
